(19) United States
(12) Patent Application Publication
(10) Pub. No.: US 2021/0380667 A1
(43) Pub. Date: Dec. 9, 2021

(54) Chymotrypsin Inhibitor Variants And The Use Thereof

(71) Applicant: Novozymes A/S, Bagsvaerd (DK)

(72) Inventors: Poul Erik Pedersen, Farum (DK); Esben Peter Friis, Herlev (DK); Carsten Andersen, Vaerloese (DK); Peter Rahbek Oestergaard, Virum (DK); Johan Qvist, Bagsvaerd (DK)

(73) Assignee: Novozymes A/S, Bagsvaerd (DK)

(21) Appl. No.: 17/284,137

(22) PCT Filed: Oct. 8, 2019
(86) PCT No.: PCT/EP2019/077229

§ 371 Date: Apr. 9, 2021

(30) Foreign Application Priority Data
Oct. 10, 2018 (EP) 18199560.6

(51) Int. Cl. C07K 14/81; C12P 21/02; C12N 9/99; C12N 9/48

(52) U.S. Cl. CPC C07K 14/811; C12N 9/48; C12N 9/99; C12P 21/02

(57) ABSTRACT

Disclosed are protease inhibitors capable of inhibiting the protease activity of a S1 or a S8 protease at ambient pH, but where the inhibitor further has a pH dependent binding to the S1 or the S8 inhibitor meaning that a complex of a S1 and a S8 protease and the inhibitor dissociates when pH is lowered to a pH below 6.0, e.g. to a pH value in the range of 4.0 to 6.0; e.g. in the range of 4.5 to 6.0; e.g. in the range of 4.5 to 5.5; whereby the protease activity is released.

### REFERENCE TO SEQUENCE LISTING

This application contains a Sequence Listing in computer readable form. The computer readable form is incorporated herein by reference.

### FIELD OF THE INVENTION

The present invention relates to variants of barley chymotrypsin Inhibitor 2a (CI-2A) and the use thereof in fermentative production of proteases

### BACKGROUND OF THE INVENTION

Proteases have for more than 30 years been important products for the enzyme industry and proteases have found use in many different commercial uses for example in detergents, for manufacturing food, and as a component in animal feed composition; to mention a few.

Proteases have the ability to degrade proteins and since proteases themselves are proteins this inherently means that proteases are self-destructive because one active protease molecule will degrade other protease molecules present in same composition and thereby reduce the active protease content of the composition. This is known in the art as autoproteolysis and various measures have been taken in order to control autoproteolysis for improving stability during production and to improve shelf life of intermediate and final protease products.

One measure to control autoproteolysis is addition protease inhibitors. It has been described to treat protease with added protease inhibitors in WO 93/20175; WO 93/13125; WO 92/05239; WO 93/17086 (Novo Nordisk) or to fuse a protease covalently with a Streptomyces SSI protease inhibitor in WO 00/01831 (Procter & Gamble); and WO 98/13483 (Procter & Gamble).

The CI-2A chymotrypsin inhibitor encoding gene of barley and the plasmid carrying the gene translated through an alfa leader sequence were described in U.S. Pat. No. 5,674,833 (1997). The CI-2A(M59P) chymotrypsin inhibitor was described in WO 92/05239 (Novo Nordisk).

The CI-2A chymotrypsin inhibitor belongs to the family 113 inhibitors according to the MEROPS classification https://www.ebi.ac.uk/merops/cgi-bin/famsum?family=I13. Several members of the family are known, mostly derived from plants, and the structure has been elucidated for some members. It inhibits serine peptidases mostly belonging to the S1 and S8 family of serine proteases.

WO 2002/016619 discloses processes for production of subtilisins, where a subtilisin (Savinase) was co-expressed with a CI-2A inhibitor leading to the production of a Savinase-CI-2A complex. The application also discloses the co-expression of Savinase with a CI-2A variant (M59P) which resulted in the production of a Savinase-CI-2A(M59P) complex that in contrast to the complex with the wild type CI-2A dissociated in detergent compositions and thereby released Savinase.

The use of inhibitors for controlling the autoproteolysis of proteases leads inevitable to the problem of dissociating the inhibitor from the protease before use of the protease for its intended purpose.

There is therefore a need to provide new inhibitors having the ability to inhibit protease activity and prevent autoproteolysis, which inhibitors can be dissociated from the protease in a simple and controllable way.

### SUMMARY OF THE INVENTION

The invention provides variants having protease inhibitor activity having at least 60% sequence identity to the mature polypeptide of SEQ ID NO: 1, and comprising a substitution at one or more positions corresponding to positions 21, 25, 26, 30, 33, 43, 44 and 52 of SEQ ID NO: 1.

The invention further provides polynucleotides encoding such variants and plasmids, expression vectors and host cells comprising such polynucleotides as well as methods for producing protease inhibitors according to the invention using such host cells.

The invention further relates to a method for producing a complex consisting of a S1 or S8 protease and a protease inhibitor variant according to the invention, comprising the steps of:
a. providing a microorganism expressing a S1 or a S8 protease, and a microorganism expressing a protease inhibitor variant;
b. cultivating the microorganism expressing a S1 or S8 protease, and the microorganism expressing a protease inhibitor variant under conditions inducing the expression of the S1 or the S8 protease and of the protease inhibitor variant, whereby a complex consisting of the S1 or the S8 protease and the protease inhibitor variant is formed; and, optionally
c. recovering the complex consisting of the S1 or S8 protease and the protease inhibitor variant from the fermentation broth.

Finally, the invention relates to a method for producing a S1 or S8 protease, comprising the steps of:
a. providing a microorganism expressing a S1 or a S8 protease, and a microorganism expressing a protease inhibitor variant according to the invention;
b. cultivating the microorganism expressing a S1 or S8 protease, and the microorganism expressing a protease inhibitor variant under conditions inducing the expression of the S1 or the S8 protease and of the protease inhibitor variant, whereby a complex consisting of the S1 or the S8 protease and the protease inhibitor variant is formed;
c. adjusting the pH to a low pH value where the complex of the S1 or S8 protease and the protease inhibitor variant dissociates and the protease activity is released, and, optionally
d. recovering the S1 or S8 protease.

### BRIEF DESCRIPTION OF DRAWINGS

FIG. 1 shows fermentation yield of Savinase comparing the yield of a Bacillus subtilis host cell expressing Savinase and the same Bacillus subtilis host cell expressing both Savinase and the CI-2A inhibitor. For more details see example 2.

FIG. 2 shows the pH dependent release of Savinase from complexes with variants of the invention compared with the CI-2A wildtype. For more details see example 4.

FIG. 3 shows the pH dependent release of a Savinase variant from complexes with variants of the invention compared with the CI-2A wildtype. For more details see example 5.

FIG. 4 shows the pH dependent release of a TY-145 variant from complexes with variants of the invention. For more details see example 6.

### BRIEF DESCRIPTION OF THE SEQUENCES

SEQ ID NO: 1 is the amino acid sequence of the Barley chymotrypsin inhibitor.

SEQ ID NO: 2 is the amino acid sequence of the S8 protease from Bacillus lentus, also known as Savinase.

SEQ ID NO: 3: is the amino acid sequence of the S8 protease from Bacillus amyloliquefaciens.

SEQ ID NO: 4 is the amino acid sequence of the S8 protease derived from a Bacillus sp., and known as subtilisin Carlsberg.

SEQ ID NO: 5: is the amino acid sequence of the S8 protease from Bacillus sp. TY145, NCIMB 40339, which was first described in WO 92/17577.

SEQ ID NO: 6: is the amino acid sequence of the S1 protease derived from Nocardiopsis sp. NRRL 18262, disclosed in WO01/58276.

### Definitions

Serine Protease: A serine protease is an enzyme which catalyzes the hydrolysis of peptide bonds, and in which there is an essential serine residue at the active site (White, Handler and Smith, 1973 “Principles of Biochemistry,” Fifth Edition, McGraw-Hill Book Company, NY, pp. 271-272).

The bacterial serine proteases have molecular weights in the 20,000 to 45,000 Dalton range. They are inhibited by diisopropylfluorophosphate. They hydrolyze simple terminal esters and are similar in activity to eukaryotic chymotrypsin, also a serine protease. A more narrow term, alkaline protease, covering a sub-group, reflects the high pH optimum of some of the serine proteases, from pH 9.0 to 11.0 (for review, see Priest (1977) Bacteriological Rev. 41 711-753).

Serine proteases of the peptidase family S1 and S8 are described in Biochem. J. 290:205-218 (1993) and in MEROPS protease database, release, 9.4 (31 Jan. 2011) (www.merops.ac.uk). The database is described in Rawlings, N.D., Barrett, A. J. & Bateman, A. (2010) ‘MEROPS: the peptidase database’, Nucleic Acids Res 38, D227-D233.

A sub-group of the serine proteases tentatively designated subtilases has been proposed by Siezen et al., Protein Engng. 4 (1991) 719-737 and Siezen et al. Protein Science 6 (1997) 501-523. They are defined by homology analysis of more than 170 amino acid sequences of serine proteases previously referred to as subtilisin-like proteases. A subtilisin was previously often defined as a serine protease produced by Gram-positive bacteria or fungi, and according to Siezen et al. now is a subgroup of the subtilases. A wide variety of subtilases have been identified, and the amino acid sequence of a number of subtilases has been determined. For a more detailed description of such subtilases and their amino acid sequences reference is made to Siezen et al. (1997).

One subgroup of the subtilases, I-S1 or “true” subtilisins, comprises the “classical” subtilisins, such as subtilisin 168 (BSS168), subtilisin BPN′, subtilisin Carlsberg (Alcalase®, Novo Nordisk A/S), and subtilisin DY (BSSDY).

A further subgroup of the subtilases, I-S2 or high alkaline subtilisins, is recognized by Siezen et al. (supra). Sub-group I-S2 proteases are described as highly alkaline subtilisins and comprises enzymes such as subtilisin PB92 (BAALKP) (Maxacal®, Gist-Brocades NV), subtilisin 309 (Savinase®, Novo Nordisk A/S), subtilisin 147 (BLS147) (Esperase®, Novo Nordisk A/S), and alkaline elastase YaB (BSEYAB).

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 18 or 19 to 83 of SEQ ID NO: 1 based on N-terminal sequencing of the mature polypeptide. It is described in the art that both amino acid N18 and L19 have been found as the N-terminal amino acid so it is therefore believed that a host cell may produce a mature polypeptide of the polypeptide having the amino acid sequence of SEQ ID NO: 1 having one of N18 and L19 as the N-terminal amino acid or it may produce a mixture of the two depending on the growth conditions and the specifically used expression construct.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

Variant: The term “variant” means a polypeptide having protease inhibitor activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the protease inhibitor activity of the mature polypeptide of SEQ ID NO: 1.

### Conventions for Designation of Variants

For purposes of the present invention, the mature polypeptide disclosed in SEQ ID NO: 1 is used to determine the corresponding amino acid residue in another protease inhibitor. The amino acid sequence of another protease inhibitor is aligned with the mature polypeptide disclosed in SEQ ID NO: 1, and based on the alignment, the amino acid position number corresponding to any amino acid residue in the mature polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another protease inhibitor can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other protease inhibitor has diverged from the mature polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to protease inhibitors capable of inhibiting the protease activity of a S1 or a S8 protease at a pH in the range of about 6.0 to about 9.0, but where the inhibitor further has a pH dependent binding to the S1 or the S8 inhibitor meaning that a complex of a S1 and a S8 protease and the inhibitor dissociates when pH is lowered to a pH below 6.0, e.g. to a pH value in the range of 4.0 to 6.0; e.g. in the range of 4.5 to 6.0; e.g. in the range of 4.5 to 5.5; whereby the protease activity is released.

Preferably the inhibitors are polypeptides.

### Variants

The present invention also relates to Protease Inhibitor variants, comprising an alteration substitutions at one or more (e.g., several) positions corresponding to positions 21, 25, 26, 30, 33, 43, 44 and 52 of the mature polypeptide of SEQ ID NO: 1, wherein the variant has protease inhibitor activity.

The present invention also provides protease inhibitor variants, comprising an alteration substitution at one or more (e.g., several) positions corresponding to 21, 25, 26, 30, 33, 43, 44 and 52, wherein variant has protease inhibitor activity.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent protease inhibitor.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 1.

In one aspect, the number of alterations in the variants of the present invention is 1-20, e.g., 1-10 and 1-5, such as 1, 2, 3, 4, 5, 6, 7, 8, 9 or 10 alterations.

In another aspect, a variant comprises an alteration substitution at one or more (e.g., several) positions corresponding to positions 21, 25, 26, 30, 33, 43, 44 and 52. In another aspect, a variant comprises an alteration at two positions corresponding to any of positions 21, 25, 26, 30, 33, 43, 44 and 52. In another aspect, a variant comprises an alteration at three positions corresponding to any of positions 21, 25, 26, 30, 33, 43, 44 and 52. In another aspect, a variant comprises an alteration at four positions corresponding to any of positions 21, 25, 26, 30, 33, 43, 44 and 52. In another aspect, a variant comprises an alteration at five positions corresponding to any of positions 21, 25, 26, 30, 33, 43, 44 and 52. In another aspect, a variant comprises an alteration at six positions corresponding to any of positions 21, 25, 26, 30, 33, 43, 44 and 52. In another aspect, a variant comprises an alteration at seven positions corresponding to any of positions 21, 25, 26, 30, 33, 43, 44 and 52. In another aspect, a variant comprises an alteration at each position corresponding to positions 21, 25, 26, 30, 33, 43, 44 and 52.

In another aspect, the variant comprises or consists of a substitution at a position corresponding to position 21. In another aspect, the amino acid at a position corresponding to position 21 is substituted with Ala, Arg, Asn, Asp, Cys, Gln, Glu, Gly, His, lie, Leu, Met, Phe, Pro, Ser, Thr, Trp, Tyr, or Val, preferably with His. In another aspect, the variant comprises or consists of the substitution K21H of the mature polypeptide of SEQ ID NO: 1.

In another aspect, the variant comprises or consists of a substitution at a position corresponding to position 25. In another aspect, the amino acid at a position corresponding to position 25 is substituted with Ala, Arg, Asn, Asp, Cys, Gln, Glu, Gly, His, lie, Leu, Lys, Met, Phe, Ser, Thr, Trp, Tyr, or Val, preferably with Ser. In another aspect, the variant comprises or consists of the substitution P25S of the mature polypeptide of SEQ ID NO: 1.

In another aspect, the variant comprises or consists of a substitution at a position corresponding to position 26. In another aspect, the amino acid at a position corresponding to position 26 is substituted with Ala, Arg, Asn, Asp, Cys, Gln, Gly, His, lie, Leu, Lys, Met, Phe, Pro, Ser, Thr, Trp, Tyr, or Val, preferably with His. In another aspect, the variant comprises or consists of the substitution E26H of the mature polypeptide of SEQ ID NO: 1.

In another aspect, the variant comprises or consists of a substitution at a position corresponding to position 30. In another aspect, the amino acid at a position corresponding to position 30 is substituted with Ala, Arg, Asn, Asp, Cys, Gln, Glu, Gly, His, lie, Leu, Met, Phe, Pro, Ser, Thr, Trp, Tyr, or Val, preferably with His. In another aspect, the variant comprises or consists of the substitution K30H of the mature polypeptide of SEQ ID NO: 1.

In another aspect, the variant comprises or consists of a substitution at a position corresponding to position 33. In another aspect, the amino acid at a position corresponding to position 33 is substituted with Ala, Arg, Asn, Asp, Cys, Gln, Gly, His, lie, Leu, Lys, Met, Phe, Pro, Ser, Thr, Trp, Tyr, or Val, preferably with His. In another aspect, the variant comprises or consists of the substitution E33H of the mature polypeptide of SEQ ID NO: 1.

In another aspect, the variant comprises or consists of a substitution at a position corresponding to position 43. In another aspect, the amino acid at a position corresponding to position 43 is substituted with Ala, Arg, Asn, Asp, Cys, Gln, Glu, Gly, His, lie, Leu, Met, Phe, Pro, Ser, Thr, Trp, Tyr, or Val, preferably with His. In another aspect, the variant comprises or consists of the substitution K43H of the mature polypeptide of SEQ ID NO: 1.

In another aspect, the variant comprises or consists of a substitution at a position corresponding to position 44. In another aspect, the amino acid at a position corresponding to position 44 is substituted with Ala, Arg, Asn, Asp, Cys, Gln, Glu, Gly, His, lie, Leu, Lys, Met, Phe, Ser, Thr, Trp, Tyr, or Val, preferably with Ala. In another aspect, the variant comprises or consists of the substitution P44A of the mature polypeptide of SEQ ID NO: 1.

In another aspect, the variant comprises or consists of a substitution at a position corresponding to position 52. In another aspect, the amino acid at a position corresponding to position 52 is substituted with Ala, Arg, Asn, Asp, Cys, Gln, Glu, Gly, His, lie, Leu, Lys, Met, Phe, Ser, Thr, Trp, Tyr, or Val, preferably with Ala. In another aspect, the variant comprises or consists of the substitution P52A of the mature polypeptide of SEQ ID NO: 1.

In another aspect, the variant comprises or consists of an alteration at positions corresponding to positions 21 and 25, such as those described above.

In another aspect, the variant comprises or consists of alterations at positions corresponding to positions 21 and 26, such as those described above.

In another aspect, the variant comprises or consists of alterations at positions corresponding to positions 21 and 30, such as those described above.

In another aspect, the variant comprises or consists of alterations at positions corresponding to positions 21 and 33, such as those described above.

In another aspect, the variant comprises or consists of alterations at positions corresponding to positions 21 and 43, such as those described above.

In another aspect, the variant comprises or consists of alterations at positions corresponding to positions 21 and 44, such as those described above.

In another aspect, the variant comprises or consists of an alteration at positions corresponding to positions 21 and 52, such as those described above.

In another aspect, the variant comprises or consists of alterations at positions corresponding to positions 25 and 26, such as those described above.

In another aspect, the variant comprises or consists of alterations at positions corresponding to positions 25 and 30, such as those described above.

In another aspect, the variant comprises or consists of alterations at positions corresponding to positions 25 and 33, such as those described above.

In another aspect, the variant comprises or consists of alterations at positions corresponding to positions 25 and 43, such as those described above.

In another aspect, the variant comprises or consists of alterations at positions corresponding to positions 25 and 44, such as those described above.

In another aspect, the variant comprises or consists of an alteration at positions corresponding to positions 25 and 52, such as those described above.

In another aspect, the variant comprises or consists of alterations at positions corresponding to positions 26 and 30, such as those described above.

In another aspect, the variant comprises or consists of alterations at positions corresponding to positions 26 and 33, such as those described above.

In another aspect, the variant comprises or consists of alterations at positions corresponding to positions 26 and 43, such as those described above.

In another aspect, the variant comprises or consists of alterations at positions corresponding to positions 26 and 44, such as those described above.

In another aspect, the variant comprises or consists of alterations at positions corresponding to positions 26 and 52, such as those described above.

In another aspect, the variant comprises or consists of an alteration at positions corresponding to positions 30 and 33, such as those described above.

In another aspect, the variant comprises or consists of alterations at positions corresponding to positions 30 and 43, such as those described above.

In another aspect, the variant comprises or consists of alterations at positions corresponding to positions 30 and 44, such as those described above.

In another aspect, the variant comprises or consists of alterations at positions corresponding to positions 30 and 52, such as those described above.

In another aspect, the variant comprises or consists of alterations at positions corresponding to positions 33 and 43, such as those described above.

In another aspect, the variant comprises or consists of alterations at positions corresponding to positions 33 and 44, such as those described above.

In another aspect, the variant comprises or consists of an alteration at positions corresponding to positions 33 and 52, such as those described above.

In another aspect, the variant comprises or consists of alterations at positions corresponding to positions 43 and 44, such as those described above.

In another aspect, the variant comprises or consists of alterations at positions corresponding to positions 43 and 52, such as those described above.

In another aspect, the variant comprises or consists of alterations at positions corresponding to positions 44 and 52, such as those described above.

In another aspect, the variant comprises or consists of alterations at positions corresponding to positions 21, 25, and 26, such as those described above.

In another aspect, the variant comprises or consists of alterations at positions corresponding to positions 21, 25, and 30, such as those described above.

In another aspect, the variant comprises or consists of alterations at positions corresponding to positions 21, 25, and 43, such as those described above.

In another aspect, the variant comprises or consists of alterations at positions corresponding to positions 21, 25, and 44, such as those described above.

In another aspect, the variant comprises or consists of alterations at positions corresponding to positions 21, 25, and 52, such as those described above.

In another aspect, the variant comprises or consists of alterations at positions corresponding to positions 21, 26, and 30, such as those described above.

In another aspect, the variant comprises or consists of alterations at positions corresponding to positions 21, 26, and 33, such as those described above.

In another aspect, the variant comprises or consists of alterations at positions corresponding to positions 21, 26, and 43, such as those described above.

In another aspect, the variant comprises or consists of alterations at positions corresponding to positions 21, 26, and 44, such as those described above.

In another aspect, the variant comprises or consists of alterations at positions corresponding to positions 21, 26, and 52, such as those described above.

In another aspect, the variant comprises or consists of alterations at positions corresponding to positions 21, 30, and 33, such as those described above.

In another aspect, the variant comprises or consists of alterations at positions corresponding to positions 21, 30, and 43, such as those described above.

In another aspect, the variant comprises or consists of alterations at positions corresponding to positions 21, 30, and 44, such as those described above.

In another aspect, the variant comprises or consists of alterations at positions corresponding to positions 21, 30, and 52, such as those described above.

In another aspect, the variant comprises or consists of alterations at positions corresponding to positions 21, 33, and 43, such as those described above.

In another aspect, the variant comprises or consists of alterations at positions corresponding to positions 21, 33, and 44, such as those described above.

In another aspect, the variant comprises or consists of alterations at positions corresponding to positions 21, 33, and 52, such as those described above.

In another aspect, the variant comprises or consists of alterations at positions corresponding to positions 21, 43, and 44, such as those described above.

In another aspect, the variant comprises or consists of alterations at positions corresponding to positions 21, 43, and 52, such as those described above.

In another aspect, the variant comprises or consists of alterations at positions corresponding to positions 21, 44, and 52, such as those described above.

In another aspect, the variant comprises or consists of alterations at positions corresponding to positions 25, 26, and 30, such as those described above.

In another aspect, the variant comprises or consists of alterations at positions corresponding to positions 25, 26, and 33, such as those described above.

In another aspect, the variant comprises or consists of alterations at positions corresponding to positions 25, 26, and 43, such as those described above.

In another aspect, the variant comprises or consists of alterations at positions corresponding to positions 25, 26, and 44, such as those described above.

In another aspect, the variant comprises or consists of alterations at positions corresponding to positions 25, 26, and 52, such as those described above.

In another aspect, the variant comprises or consists of alterations at positions corresponding to positions 25, 30, and 33, such as those described above.

In another aspect, the variant comprises or consists of alterations at positions corresponding to positions 25, 30, and 43, such as those described above.

In another aspect, the variant comprises or consists of alterations at positions corresponding to positions 25, 30, and 44, such as those described above.

In another aspect, the variant comprises or consists of alterations at positions corresponding to positions 25, 30, and 52, such as those described above.

In another aspect, the variant comprises or consists of alterations at positions corresponding to positions 25, 33, and 43, such as those described above.

In another aspect, the variant comprises or consists of alterations at positions corresponding to positions 25, 33, and 44, such as those described above.

In another aspect, the variant comprises or consists of alterations at positions corresponding to positions 25, 33, and 52, such as those described above.

In another aspect, the variant comprises or consists of alterations at positions corresponding to positions 25, 43, and 44, such as those described above.

In another aspect, the variant comprises or consists of alterations at positions corresponding to positions 25, 43, and 52, such as those described above.

In another aspect, the variant comprises or consists of alterations at positions corresponding to positions 25, 44, and 52, such as those described above.

In another aspect, the variant comprises or consists of alterations at positions corresponding to positions 26, 30, and 33, such as those described above.

In another aspect, the variant comprises or consists of alterations at positions corresponding to positions 26, 30, and 43, such as those described above.

In another aspect, the variant comprises or consists of alterations at positions corresponding to positions 26, 30, and 44, such as those described above.

In another aspect, the variant comprises or consists of alterations at positions corresponding to positions 26, 30, and 52, such as those described above.

In another aspect, the variant comprises or consists of alterations at positions corresponding to positions 26, 33, and 43, such as those described above.

In another aspect, the variant comprises or consists of alterations at positions corresponding to positions 26, 33, and 44, such as those described above.

In another aspect, the variant comprises or consists of alterations at positions corresponding to positions 26, 33, and 52, such as those described above.

In another aspect, the variant comprises or consists of alterations at positions corresponding to positions 26, 43, and 44, such as those described above.

In another aspect, the variant comprises or consists of alterations at positions corresponding to positions 26, 43, and 52, such as those described above.

In another aspect, the variant comprises or consists of alterations at positions corresponding to positions 26, 44, and 52, such as those described above.

In another aspect, the variant comprises or consists of alterations at positions corresponding to positions 30, 33, and 43, such as those described above.

In another aspect, the variant comprises or consists of alterations at positions corresponding to positions 30, 33, and 44, such as those described above.

In another aspect, the variant comprises or consists of alterations at positions corresponding to positions 30, 33, and 52, such as those described above.

In another aspect, the variant comprises or consists of alterations at positions corresponding to positions 30, 43, and 44, such as those described above.

In another aspect, the variant comprises or consists of alterations at positions corresponding to positions 30, 43, and 52, such as those described above.

In another aspect, the variant comprises or consists of alterations at positions corresponding to positions 30, 44, and 52, such as those described above.

In another aspect, the variant comprises or consists of alterations at positions corresponding to positions 33, 43, and 44, such as those described above.

In another aspect, the variant comprises or consists of alterations at positions corresponding to positions 33, 43, and 52, such as those described above.

In another aspect, the variant comprises or consists of alterations at positions corresponding to positions 33, 44, and 52, such as those described above.

In another aspect, the variant comprises or consists of alterations at positions corresponding to positions 43, 44, and 52, such as those described above.

In another aspect, the variant comprises or consists of one or more (e.g., several) substitutions selected from the group consisting of K21H, P25S, E26H, K30H, E33H, K43H, P44A and P52A.

In another aspect, the variant comprises or consists of the substitutions K21H+E26H of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+K30H of the mature polypeptide of SEQ ID NO: 2, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+K30H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K30H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K30H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+K30H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+K30H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+K30H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+K30H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+K30H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K30H+E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+K30H+E33H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+K30H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+K30H+E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions E26H+K30H+E33H+K43H of the mature polypeptide of SEQ ID NO: 1, or of a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 1 which has protease inhibitor activity, and further the variant dissociates from the protease at a higher pH compared to the mature inhibitor of SEQ ID NO: 1.

In another aspect, the variant comprises or consists of the substitutions K21H+E26H+K30H+H33H+K43H of the mature polypeptide of SEQ ID NO: 1.

The variants may further comprise one or more additional alterations at one or more (e.g., several) other positions.

The amino acid changes may be of a minor nature, that is conservative amino acid substitutions or insertions that do not significantly affect the folding and/or activity of the protein; small deletions, typically of 1-10 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease inhibitor activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The variants may consist of 60 to 90 amino acids, e.g., 70 to 85, and 75 to 83 amino acids.

Preferred examples of variants according to the inventions include variants comprising the substitutions using SEQ ID NO: 1 for numbering:
K43H+P44A+P52A;
K21H+P25S+K30H;
K21H+P25S+E26H+K30H+E33H; and
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; and
K21H+P25S+E26H+K30H+E33H+E45G+Q47H;

and having at least 60% sequence identity to SEQ ID NO: 1.

Other examples include variants comprising the substitutions using SEQ ID NO: 1 for numbering:

P25S+P44A+P52A+P80V;

P25S+P44A+P52A+P801;

K21H+E26H+D41N;

E33H;

K43H+E45G,

K43H+E45A;

Q47H;

149H; and

L73H.

Particular preferred variants according to the inventions include variants having the sequence of SEQ ID NO: 1 with following substitutions:
K43H+P44A+P52A;
K21H+P25S+K30H;
K21H+P25S+E26H+K30H+E33H; and
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; and
K21H+P25S+E26H+K30H+E33H+E45G+Q47H.

The variants of the invention has the ability to dissociate from a complex consisting of the variant and a S8 or S1 protease e.g. the Bacillus lentus protease having SEQ ID NO: 2 (also known as Savinase) when pH is lowered to a pH value below 6, e.g. to a pH of 4.5.

The skilled person will appreciate that the exact pH value where complex consisting of a S1 or a S8 protease and a variant of the invention dissociates, depends on the specific sequence of both the S1 or the S8 protease and the particular variant of the invention, but for a complex consisting of a given S1 or S8 protease and a variant of the invention the complex dissociates at a pH value that is at least 0.4 pH unit, preferably at least 0.5 pH unit, preferably at least 0.6 pH unit, preferably at least 0.7 pH unit, preferably at least 0.8 pH unit, preferably at least 0.9 pH unit, or preferably at least 1.0 pH unit higher than the pH where a complex consisting of the same S1 or S8 protease and the wildtype CI-2A inhibitor dissociates.

This can conveniently be analysed by following procedure:
a) Prepare a complex of an S8 or an S1 protease and a protease inhibitor variant of the invention by mixing equimolar amounts of the protease and the protease inhibitor;
b) Incubate aliquots of the complex prepared in a) in buffer solutions at various pH values in the range of 6 to 3.5; e.g. at pH 6, 5.5, 5.0, 4.5, 4.0 and 3.5 at a temperature in the range of 25-50°;
c) After 60 minutes incubation analyse the protease activities in each sample using a standard protease assay; and
d) Repeat the steps a) to c) with wildtype CI-2A as reference.

A preferred assay for determining the ability of the variants to dissociate from a protease is the following procedure:
a) Prepare a complex of a given protease inhibitor variant of the invention and Savinase, by mixing equimolar amounts of Savinase and the protease inhibitor;
b) Incubate aliquots of the complex prepared in a) in buffer solutions at various pH values in the range of 6 to 3.5; e.g. at pH 6, 5.5, 5.0, 4.5, 4.0 and 3.5 at a temperature in the range of 25-50°;
c) After 60 minutes incubation analyse the protease activities in each sample using a standard protease assay; and
d) Repeat the steps a) to c) with wildtype CI-2A as reference.
   This assay is exemplified in Example 3.

For the variants of the invention it can be seen that the protease activity peaks at least 0.5 pH unit higher compared with the wildtype CI-2A, preferably at a pH in the range of 4.0 to 4.5.

The wildtype CI-2A can also be released when the pH is lowered, but this requires that the pH is lowered to a much lower pH value, such as below 4.0 or even below 3.5 before the inhibitor is released or denatured due to the low pH.

Thus, the variants has the ability to inhibit S1 and S8 proteases at pH values in the neutral to alkaline area, typically in pH values in the range of 5.5 to 9.0, and further the variants can easily be dissociated from the S1 and S8 proteases by lowering the pH to a pH value in the range of 5.5 to 4.0, and thereby release the S1 and S8 protease.

This has the benefit that the variants of the invention can be added to a composition comprising one or more S1 and/or S8 at a neutral to alkaline pH value and thereby inhibit the protease activity so the proteases in the composition is protected against autoproteolysis and other proteins in the composition is protected against protease degradation, and further can the pH activity be released when desired by lowering the pH.

The wildtype CI-2A inhibitor can also inhibit the S1 and S8 proteases and protect against autoproteolysis and protease degradation but the wild type CI-2SA can not be removed from the S1 and S8 proteases as easily as the variants of the invention. Wild type CI-2A requires that the pH is lowered at least 0.5 pH units lower than the pH unit required for the variants. When pH is lower so much as required to release the wild-type CI-2A inhibitor from the S1 and/or S8 protease that a significant fraction of the S1 and/or S8 protease present will be denatured due to the low pH conditions, but using the variants of the invention the pH need not to be lowered to such low pH values as required to dissociate the complex with the wild type CI-2A and therefore will the loss of protease activity be significantly reduced using the variants of the invention.

Therefore, may the variants of the invention be used to protect S1 and/or S8 proteases against autoproteolysis and to protect proteins in a composition comprising one or more S1 and/or S8 proteases and one or more additional proteases against proteolysis at pH in the neutral and basic area, where the protease-inhibitor complexes can easily dissociate and the protease activity be released by lowering the pH when desired.

Use of Variants of the Invention

The present invention also relates to methods for production, recovery and purification of S1 and/or S8 proteases and/or complexes of inhibitors of the invention, preferably variants of the invention and S1 and/or S8 proteases.

In one preferred embodiment, the invention relates to production and recovery of a S1 or S8 protease or of a complex consisting of a S1 or S8 protease and a variant of the invention. In this embodiment, a complex consisting of a S1 or S8 protease and a variant of the invention is prepared by a fermentation process where the S1 or S8 protease and the variant of the invention is produced and secreted into the fermentation broth. The fermentation may be a fermentation where a host cell expresses both the S1 or S8 protease and the variant of the invention, or it may be a fermentation process where a microorganism that expresses a S1 or S8 protease is co-cultivated with a microorganism that expresses a variant of the invention. Preferably, the S1 or S8 protease and the variant of the invention is expressed in approximately equimolar amounts such as in molar ratio in the range of 1:3 to 3:1; e.g. in molar ratio in the range of 1:2 to 2:1; e.g. in molar ratio in the range of 1:1.5 to 1.5:1; e.g. in molar ratio in the range of 1:1.3 to 1.3:1; e.g. in molar ratio in the range of 1:1.2 to 1.2:1; e.g. in molar ratio in the range of 1:1.1 to 1.1:1; or in a molar ration of approximately 1:1.

The fermentation should take place at a pH value where the S1 or S8 protease and the variant of the invention forms complexes, typically at a pH value above 5.5, such as in the range of pH 5.5 to 9, e.g. in the range of 6.0 to 9.0.

When the fermentation is complete complexes consisting of a S1 or a S8 protease and a variant of the invention is recovered by a series of separation steps as known in the art such as filtration, centrifugation, precipitations, microfiltration, concentration etc.

In some embodiments, the complexes consisting of a S1 or S8 protease and a variant of the invention are present in precipitated form in the fermentation broth after the fermentation process. In such embodiments technologies for recovering enzymes precipitated during fermentation such as disclosed in WO2008/110498, PCT/EP2018/058387 (14385 published early October) and WO2003/050274 may be used for the recovery process.

If it is desired to recover the free S1 or S8 protease without the variant of the invention a low pH step should be included in the recovery process in order to dissociate the complexes of S1 or S8 protease and the variant of the invention. The low pH step for dissociating the complexes may be performed immediately after the fermentation, or it may be performed after the recovery process where the complexes have been recovered from the fermentation broth or it may be performed at any stage during the recovery process.

Fermenting one or more S1 and/or S8 proteases and one or more variants of the invention in same fermenter has the benefit that the S1 and/or S8 proteases are completely or partially protected against proteolysis which typically means that the yield of S1 and/or S8 protease is increased.

After recovery the S1 and/or S8 protease may be used as known in the art.

S1 and/or S8 Proteases

Family S1 is the largest of all of the peptidase families, by both the number of proteins that have been sequenced and the number of distinct peptidase activities. The peptidases of family S1 contain the catalytic triad His, Asp and Ser found in all members of subclan PA/S. Activity has been claimed for human testes-specific protein TSP50 which has the serine replaced by threonine, although the serine is conserved for this protein from many other vertebrates. Chymotrypsin is an example of an well known S1 protease.
Peptidase family S8 contains the serine endopeptidase subtilisin and its homologues. Members of family S8 have a catalytic triad in the order Asp, His and Ser in the sequence, which is a different order to that of families S1. In subfamily S8A, the active site residues frequently occurs in the motifs Asp-Thr/Ser-Gly, His-Gly-Thr-His and Gly-Thr-Ser-Met-Ala-Xaa-Pro. In subfamily S8B, the catalytic residues frequently occur in the motifs Asp-Asp-Gly, His-Gly-Thr-Arg and Gly-Thr-Ser-Ala/Val-Ala/Ser-Pro.
Until the determination of the sequence and structure of subtilisin, it was thought that all serine-type peptidases would be homologous to chymotrypsin. Subtilisin was clearly very different and unrelated to chymotrypsin. Family S8, also known as the subtilase family, is the second largest family of serine peptidases, both in terms of number of sequences and characterized peptidases. The family is divided into two subfamilies, with subtilisin the type-example for subfamily S8A and kexin the type-example for subfamily S8B.
Examples of S1 proteases according to the invention include 10R protease as well as protease having at least 60% sequence identity to 10R, e.g at least 70% sequence identity; e.g. at least 80% sequence identity, e.g. at least 90% sequence identity, e.g. at least 95% sequence identity, e.g. at least 96% sequence identity, e.g. at least 97% sequence identity, e.g. at least 98% sequence identity, e.g. at least 99% sequence identity to SEQ ID NO: 6.
Examples of S8 proteases according to the invention include subtilases, subfamily S8A, such as the protease derived from Bacillus amyloliquefaciens and having the sequence of SEQ ID NO: 3, the protease derived from Bacillus lentus and having the sequence of SEQ ID NO: 2; the Bacillus sp. protease known as subtilisin Carlsberg and having the sequence of SEQ ID NO: 4 and the protease derived from the Bacillus sp. TY145 and having the sequence of SEQ ID NO: 5 as well as subtilases having at least 60% sequence identity to one these, e.g at least 70% sequence identity; e.g. at least 80% sequence identity, e.g. at least 90% sequence identity, e.g. at least 95% sequence identity, e.g. at least 96% sequence identity, e.g. at least 97% sequence identity, e.g. at least 98% sequence identity, e.g. at least 99% sequence identity to SEQ ID NO: 2, 3, 4 or 5.

### Nucleic Acid Sequences

The present invention also relates to an isolated nucleic acid sequence, which encodes a protease inhibitor variant of the present invention.

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used.

An isolated nucleic acid sequence can, for example, be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the subtilase, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

For purposes of the present invention, the degree of identity between two nucleic acid sequences is determined as described above.

Modification of a nucleic acid sequence encoding a subtilase of the present invention may be necessary for the synthesis of subtilases substantially similar to the subtilase. The term “substantially similar” to the subtilase refers to non-naturally occurring forms of the subtilase. These subtilases may differ in some engineered way from the subtilase isolated from its native source, e.g., variants that differ in specific activity, thermostability, pH optimum, or the like. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active subtilase. Amino acid residues essential to the activity of the polypeptide encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for proteolytic activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

### Nucleic Acid Constructs

The present invention also relates to nucleic acid constructs comprising a nucleic acid sequence of the present invention operably linked to one or more control sequences capable of directing the expression of the polypeptide in a suitable host cell.

An isolated nucleic acid sequence encoding a protease inhibitor complex of the present invention may be manipulated in a variety of ways to provide for expression of the subtilase. Manipulation of the nucleic acid sequence prior to its insertion into a vector may be desirable or necessary depending on the expression vector. The techniques for modifying nucleic acid sequences utilizing recombinant DNA methods are well known in the art.

The control sequences include all components which are necessary or advantageous for the expression of a subtilase of the present invention. Each control sequence may be native or foreign to the nucleic acid sequence encoding the subtilase. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a subtilase.

The control sequence may be an appropriate promoter sequence, a nucleic acid sequence which is recognized by a host cell for expression of the nucleic acid sequence. The promoter sequence contains transcriptional control sequences which mediate the expression of the subtilase. The promoter may be any nucleic acid sequence which shows transcriptional activity in the host cell of choice including mutant, truncated, and hybrid promoters, and may be obtained from genes encoding extracellular or intracellular subtilases either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

The control sequence may also be a suitable transcription terminator sequence, a sequence recognized by a host cell to terminate transcription. The terminator sequence is operably linked to the 3′ terminus of the nucleic acid sequence encoding the subtilase. Any terminator which is functional in the host cell of choice may be used in the present invention.

The control sequence may also be a suitable leader sequence, a nontranslated region of an mRNA which is important for translation by the host cell. The leader sequence is operably linked to the 5′ terminus of the nucleic acid sequence encoding the polypeptide. Any leader sequence that is functional in the host cell of choice may be used in the present invention.

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region that codes for an amino acid sequence linked to the amino terminus of a subtilase and directs the encoded subtilase into the cell's secretory pathway. The 5′ end of the coding sequence of the nucleic acid sequence may inherently contain a signal peptide coding region naturally linked in translation reading frame with the segment of the coding region which encodes the secreted subtilase. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the subtilase. However, any signal peptide coding region which directs the expressed subtilase into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis betalactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding region that codes for an amino acid sequence positioned at the amino terminus of a subtilase. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a subtilase, the propeptide region is positioned next to the amino terminus of a subtilase and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

### Expression Vectors

A recombinant expression vector comprising a DNA construct encoding the enzyme of the invention may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome in part or in its entirety and replicated together with the chromosome(s) into which it has been integrated.

The vector is preferably an expression vector in which the DNA sequence encoding the enzyme of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the enzyme.

The promoter may be any DNA sequence which shows transcriptional activity in the host cell of choice and may be derived from genes encoding proteins either homologous or heterologous to the host cell.

Examples of suitable promoters for use in bacterial host cells include the promoter of the Bacillus stearothermophilus maltogenic amylase gene, the Bacillus licheniformis alphaamylase gene, the Bacillus amyloliquefaciens alpha-amylase gene, the Bacillus subtilis alkaline protease gen, or the Bacillus pumilus xylosidase gene, or the phage Lambda P_{R} or P_{L} promoters or the E. coli lac, trp or tac promoters.

The DNA sequence encoding the enzyme of the invention may also, if necessary, be operably connected to a suitable terminator.

The recombinant vector of the invention may further comprise a DNA sequence enabling the vector to replicate in the host cell in question.

The vector may also comprise a selectable marker, e.g. a gene the product of which complements a defect in the host cell, or a gene encoding resistance to e.g. antibiotics like kanamycin, chloramphenicol, erythromycin, tetracycline, spectinomycine, or the like, or resistance to heavy metals or herbicides.

To direct an enzyme of the present invention into the secretory pathway of the host cells, a secretory signal sequence (also known as a leader sequence, prepro sequence or pre sequence) may be provided in the recombinant vector. The secretory signal sequence is joined to the DNA sequence encoding the enzyme in the correct reading frame. Secretory signal sequences are commonly positioned 5′ to the DNA sequence encoding the enzyme. The secretory signal sequence may be that normally associated with the enzyme or may be from a gene encoding another secreted protein.

The procedures used to ligate the DNA sequences coding for the present enzyme, the promoter and optionally the terminator and/or secretory signal sequence, respectively, or to assemble these sequences by suitable PCR amplification schemes, and to insert them into suitable vectors containing the information necessary for replication or integration, are well known to persons skilled in the art (cf., for instance, Sambrook et al., op.cit.).

### Host Cell

The DNA sequence encoding the present enzyme introduced into the host cell may be either homologous or heterologous to the host in question. If homologous to the host cell, i.e. produced by the host cell in nature, it will typically be operably connected to another promoter sequence or, if applicable, another secretory signal sequence and/or terminator sequence than in its natural environment. The term “homologous” is intended to include a DNA sequence encoding an enzyme native to the host organism in question. The term “heterologous” is intended to include a DNA sequence not expressed by the host cell in nature. Thus, the DNA sequence may be from another organism, or it may be a synthetic sequence.

The host cell into which the DNA construct or the recombinant vector of the invention is introduced may be any cell which is capable of producing the present enzyme and includes bacteria, yeast, fungi and higher eukaryotic cells.

Examples of bacterial host cells which, on cultivation, are capable of producing the enzyme of the invention are gram-positive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gram-negative bacteria such as Escherichia coli. The transformation of the bacteria may be effected by protoplast transformation, electroporation, conjugation, or by using competent cells in a manner known per se (cf. Sambrook et al., supra).

When expressing the enzyme in bacteria such as E. coli, the enzyme may be retained in the cytoplasm, typically as insoluble granules (known as inclusion bodies), or may be directed to the periplasmic space by a bacterial secretion sequence. In the former case, the cells are lysed and the granules are recovered and denatured after which the enzyme is refolded by diluting the denaturing agent. In the latter case, the enzyme may be recovered from the periplasmic space by disrupting the cells, e.g. by sonication or osmotic shock, to release the contents of the periplasmic space and recovering the enzyme.

When expressing the enzyme in gram-positive bacteria such as Bacillus or Streptomyces strains, the enzyme may be retained in the cytoplasm, or may be directed to the extracellular medium by a bacterial secretion sequence. In the latter case, the enzyme may be recovered from the medium as described below.

Method of Producing Protease and/or Protease Inhibitor Complex

The present invention provides a method of producing an isolated protease and/or variant inhibitor complex according to the invention, wherein a suitable host cell, which has been transformed with a DNA sequence encoding the protease and/or variant inhibitor complex, is cultured under conditions permitting the production of the complex, and the resulting complex or protease is recovered from the culture.

When an expression vector comprising a DNA sequence encoding the protein is transformed into a heterologous host cell it is possible to enable heterologous recombinant production of the protein of the invention.

Thereby it is possible to make a highly purified subtilase composition, characterized in being free from homologous impurities.

In this context homologous impurities mean any impurities (e.g. other polypeptides than the complex or protease of the invention) that originate from the homologous cell, from where the protein of the invention is originally obtained.

The medium used to culture the transformed host cells may be any conventional medium suitable for growing the host cells in question. The expressed subtilase complex may conveniently be secreted into the culture medium and may be recovered therefrom by wellknown procedures including separating the cells from the medium by centrifugation or filtration, precipitating proteinaceous components of the medium by means of a salt such as ammonium sulphate, followed by chromatographic procedures such as ion exchange chromatography, affinity chromatography, or the like.

### Preferred Embodiments

The invention is further described by way of the following numbered embodiments:

Embodiment 1. A protease inhibitor having the ability to inhibit a S1 or a S8 protease at pH in the range of 6.0 to 9.0, where a complex of a S1 or S8 protease and the inhibitor dissociates when pH is lowered to a pH below 6.0.

Embodiment 2. The protease inhibitor of embodiment 1, where the inhibitor is a polypeptide.

Embodiment 3. The protease inhibitor of embodiment 1 or 2, wherein the complex of a S1 or S8 protease and the inhibitor dissociates when pH is adjusted to a pH value in the range of 4.0 to 6.0.

Embodiment 4. The protease inhibitor of embodiment 3, wherein the complex of a S1 or S8 protease and the inhibitor dissociates when pH is adjusted to a pH value in the range of 4.5 to 6.0.

Embodiment 5. The protease inhibitor of embodiment 4, wherein the complex of a S1 or S8 protease and the inhibitor dissociates when pH is adjusted to a pH value in the range of 4.5 to 5.5.

Embodiment 6. The protease inhibitor according to embodiment 2, wherein the protease inhibitor is a variant having protease inhibitor activity and having at least 60% sequence identity to the mature polypeptide of SEQ ID NO: 1, and comprising a substitution at one or more positions corresponding to positions 21, 25, 26, 30, 33, 43, 44 and 52 of SEQ ID NO: 1.

Embodiment 7. The variant of embodiment 6, wherein a complex comprising the variant and the protease having the sequence of SEQ ID NO: 2 dissociates at a higher pH compared with a complex comprising the protease inhibitor having the sequence of the mature polypeptide of SEQ ID NO: 1 and the protease having the sequence of SEQ ID NO: 2.

Embodiment 8. The variant of embodiment 7, wherein the complex comprising the variant and the protease having the sequence of SEQ ID NO: 2 dissociates at a pH value that is at least 0.4 pH units higher, e.g. at least 0.5 pH units higher; e.g. at least 0.6 pH units higher; e.g. at least 0.7 pH units higher; e.g. at least 0.8 pH units higher; e.g. at least 0.9 pH units higher; e.g. at least 1.0 pH units higher than the pH value where complex comprising the protease inhibitor having the sequence of the mature polypeptide of SEQ ID NO: 1 and the protease having the sequence of SEQ ID NO: 2 dissociates.

Embodiment 9. The variant according to any of the embodiments 6-8, wherein the substitutions are selected among substitutions corresponds to following substitutions in SEQ ID NO: 1: K21H, P25S, E26H, K30H, E33H, K43H, P44A and P52A.

Embodiment 10. The variants according to any of the embodiments 6-9, comprising the substitutions: K21H, P25S, E26H, K39H and E33H.

Embodiment 11. The variants of embodiment 10, further comprising one or more substitutions selected among: D42N, E45G and Q47H.

Embodiment 12. The variants according to any of the embodiments 6 to 9, comprising the substitutions:
K43H+P44A+P52A;
K21H+P25S+K30H;
K21H+P25S+E26H+K30H+E33H;
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; and
K21H+P25S+E26H+K30H+E33H+E45G+Q47H.

Embodiment 13. The variants of embodiment 12, having the sequence of SEQ ID NO: 1 with the substitutions:
K43H+P44A+P52A;
K21H+P25S+K30H;
K21H+P25S+E26H+K30H+E33H;
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; or
K21H+P25S+E26H+K30H+E33H+E45G+Q47H.

Embodiment 14. A polynucleotide encoding a protease inhibitor according to any of the embodiments 1-13.

Embodiment 15. A plasmid, expression construct or host cell comprising the polynucleotide of embodiment 14.

Embodiment 16. A method of producing a protease inhibitor according to any of the embodiments 1-13 comprising the steps of:
a. providing a host cell of embodiment 15,
b. cultivating the host cell under conditions leading to expression of the protease variant, producing a fermentation broth; and
c. recovering the protease inhibitor variant from the fermentation broth

Embodiment 17. A method for producing a complex consisting of a S1 or S8 protease and a protease inhibitor according to embodiments 1-13, comprising the steps of:
a. providing a microorganism expressing a S1 or a S8 protease, and a microorganism expressing a protease inhibitor according to embodiments 1-13;
b. cultivating the microorganism expressing a S1 or S8 protease, and the microorganism expressing a protease inhibitor variant under conditions inducing the expression of the S1 or the S8 protease and of the protease inhibitor variant, whereby a complex consisting of the S1 or the S8 protease and the protease inhibitor variant is formed; and
c. recovering the complex consisting of the S1 or S8 protease and the protease inhibitor variant from the fermentation broth.

Embodiment 18. The method of embodiment 17, wherein the microorganism expressing the S1 or S8 protease is the same microorganism that expresses the protease inhibitor.

Embodiment 19. A method for producing a S1 or S8 protease, comprising the steps of:
a. providing a microorganism expressing a S1 or a S8 protease, and a microorganism expressing a protease inhibitor according to embodiments 1-13;
b. cultivating the microorganism expressing a S1 or S8 protease, and the microorganism expressing a protease inhibitor variant under conditions inducing the expression of the S1 or the S8 protease and of the protease inhibitor variant, whereby a complex consisting of the S1 or the S8 protease and the protease inhibitor variant is formed;
c. adjusting the pH to a low pH value where the complex of the S1 or S8 protease and the protease inhibitor variant dissociates and the protease activity is released, and
d. recovering the S1 or S8 protease.

Embodiment 20. The method of embodiment 19, where pH in step c. is adjusted to a pH value in the range of 4.0 to 4.5.

Embodiment 21. The method according to embodiment 19 or 20, wherein the microorganism expressing the S1 or S8 protease is the same microorganism that expresses the protease inhibitor.

Embodiment 22. The method according to any of the embodiments 19 to 21, wherein step c. is performed before the recovery process, during the recovery process or after the complex consisting of the S1 or the S8 protease and the protease inhibitor variant has been recovered.

Embodiment 23. The method according to any of the embodiments 17 to 22, wherein the S1 or the S8 protease is selected among polypeptides having protease activity and having at least 60% sequence identity, e.g. at least 70% sequence identity; having at least 80% sequence identity, e.g. at least 90% sequence identity; having at least 95% sequence identity, e.g. at least 96% sequence identity; having at least 97% sequence identity, e.g. at least 98% sequence identity to one of SEQ ID NO: 2; SEQ ID NO: 3; SEQ ID NO: 4, SEQ ID NO: 5 or SEQ ID NO: 6.

### EXAMPLES

Materials and Methods

Buffers:

Universal buffers: 100 mM succinic acid, 100 mM HEPES, 100 mM CHAS, 100 mM CABS, 1 mM CaCl2, 150 mM KCl, 0.01% Triton X-100, pH 2.0-6.0

### Example 1 Preparation of CI-2A Variants

CI-2A variants were generated and a B. subtilis host cell comprising a gene encoding B. lentus protease (Savinase) having the amino acid sequence of SEQ ID NO: 2, and a gene encoding the variant using technologies essentially as disclosed in Example 1 of WO 2002/016619. Following variants of the CI-2A variants having the sequence of the mature polypeptide of SEQ ID NO: 1 with the substitutions:
K43H+P44A+P52A;
K21H+P25S+K30H;
K21H+P25S+E26H+K30H+E33H;
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; and
K21H+P25S+E26H+K30H+E33H+E45G+Q47H.

The host strains expressing Savinase and the variants were plated on protease sensitive plates, where they did not give rise to clearing zones around the colonies indicating that the strains did not secrete active protease. A control strain producing Savinase without a CI2A variant had clearing zones around the colonies. This experiment shows that the generated variants were active and were capable of inhibiting the produced Savinase and thereby preventing formation of clearing zones.

### Example 2. Co-Expression of Savinase and CI-2A

A Bacillus subtilis host strain was transformed with 3 copies of B. lentus protease having SEQ ID NO: 2 (Savinase), and further transformed with 2 copies of the CI-2A wildtype having the amino acid sequence of the mature protein of SEQ ID NO:1.

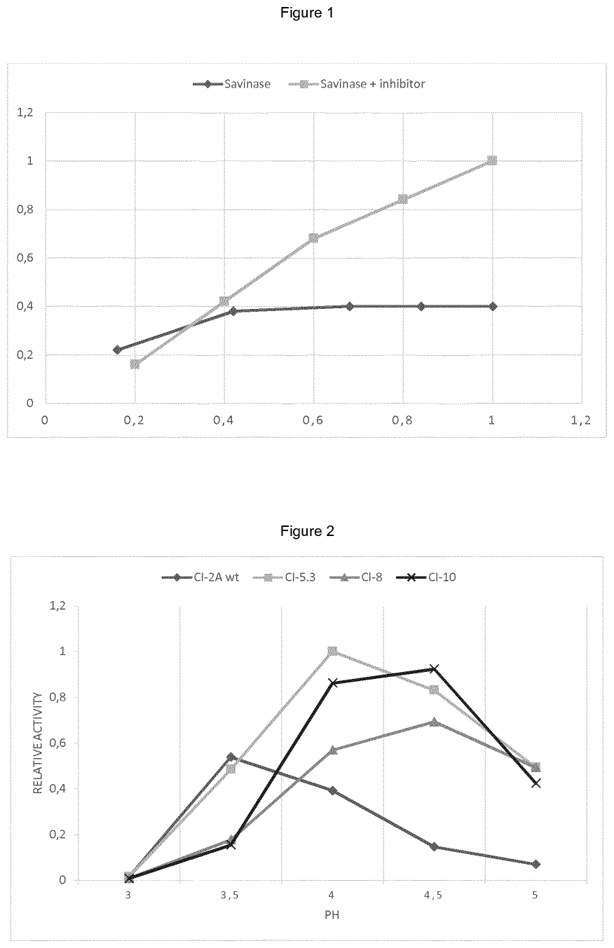

The strain comprising 3 copies of B. lentus protease and the strain comprising 3 copies of B. lentus protease and 2 copies of the CI-2A inhibitor were each fermented in a lab scale fermenter at 37° C. and the protease yield were recorded and shown in FIG. 1.

The result shows that the protease level in the fermentation broth of the strain expressing the B. lentus protease without the CI-2A inhibitor reached a plateau before half of the fermentation time, whereas the strain expressing the B. lentus protease and the CI-2A inhibitor continues to growth throughout the whole fermentation and reached a level more than 2 times the level reached for the strain without the inhibitor.

### Example 3. Co-Expression of Savinase and CI-2A Variants and Release of Protease Activity

A Bacillus subtilis host strain was transformed with 3 copies of B. lentus protease having SEQ ID NO: 2 (Savinase), and further transformed with 2 copies of the CI-2A variants having the amino acid sequence of the mature protein of SEQ ID NO:1 with substitutions:
(CI5.3) K21H+P25S+K30H;
(CI08) K21H+P25S+E26H+K30H+E33H; or
(CI10) K30H+E33H+K43H+P44A+P52A.

The strains were prepared essentially as described in example 2.

The strains 3 copies of B. lentus protease and 2 copies of the CI-2A variants were each fermented in a lab scale fermenter at 37° C., the protease yield assessed and it was found that it developed essentially as for the CI-2A wildtype inhibitor as shown in FIG. 1.

### Example 4. Release of Protease Activity from a Complex of Savinase and Variants of the Invention

The fermentation broths from example 3 were tested for release of protease activity at different pH values using following procedure:
The culture broths were centrifuged at 14.000 g for 3 minutes and the supernatant recovered; 50 μl supernatants were mixed with 200 μl Universal Buffer adjusted to pH 3.0, 3.5, 4.0, 4.5 and 5.0 and incubated 1 hour at 30° C.;
Each incubation mixture was diluted 50× in 0.01% Triton and protease activity measured in Universal buffer, pH9.0 using Suc-AAPF-pNA as substrate

The results are shown in FIG. 2. The results show that for the CI-2A wildtype and also for each variant the protease activity starts at a low level at pH 5, and the protease level rises as pH is lowered because protease is released from the inhibitor. When the pH is below 4-4.5 the protease activity falls again presumably due to denaturation of the protease enzyme at such low pH values and all protease activity is lost when pH comes down at 3.0 For the variants it can be seen that the protease activity peak at a higher pH value, between 4 and 4.5, than for the CI-2A wildtype, peaking around pH 3.5, indicating that the variants are dissociating from the protease at a higher pH than the wildtype CI-2A. Further, it can be seen that the protease activity for the variants are significantly higher than for the wildtype CI-2A, presumably because the protease is not very stable at the very low pH necessary to dissociate the wildtype CI-2A inhibitor from the protease.

### Example 5. Co-Expression of a Savinase Variant and CI-2A Variants and Release of Protease Activity

A Bacillus subtilis host strain was transformed with 3 copies of B. lentus protease variants having the amino acid sequence of SEQ ID NO: 2 with the substitutions S9E N43R N76D V205I Q206L Y209W S259D N261W L262E (using SEQ ID NO: 3 for numbering, disclosed in WO 2016/087617), and further transformed with 2 copies of the CI-2A variants having the amino acid sequence of the mature protein of SEQ ID NO:1 with substitutions:
(CI08) K21H+P25S+E26H+K30H+E33H; or
(CI10) K30H+E33H+K43H+P44A+P52A.

The strains were prepared essentially as described in example 2.

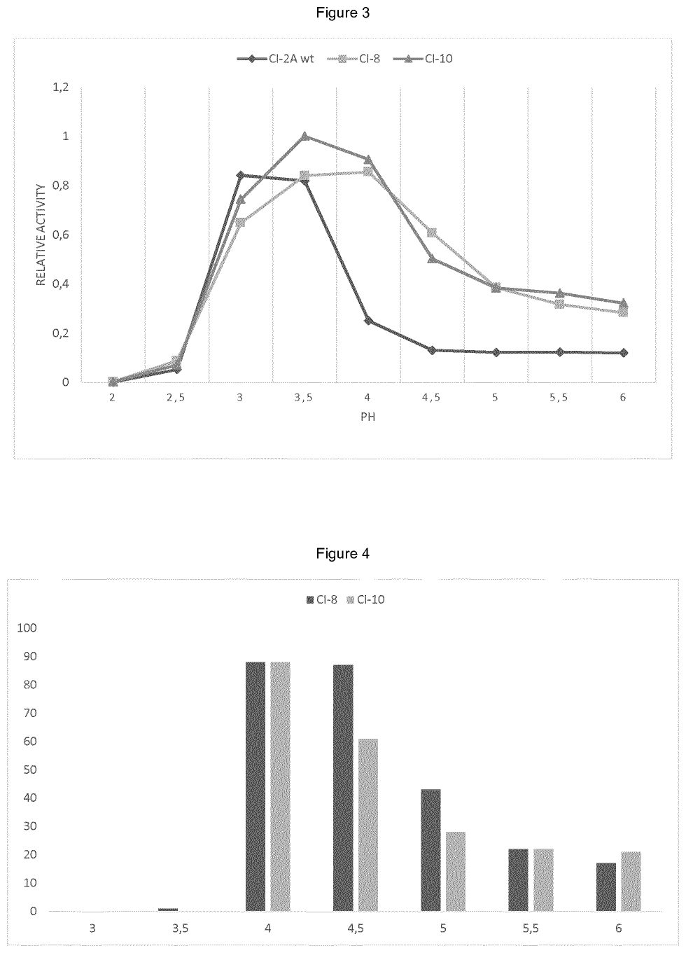

The fermentation broths were tested for release of protease activity at different pH values, essentially as described in example 4 except that pH values between 2.0 and 6.0 were used. Results are shown in FIG. 3. The results show that the protease activity in the fermentation broth with the variants of the invention peak at a higher pH value, between 3.5 and 4.0, than for the CI-2A wildtype, peaking around pH 3.0, indicating that the variants are dissociating from the protease at a higher pH than the wildtype CI-2A. At pH below 3 the protease activity is quickly lost due to inactivation under such harsh conditions.

### Example 6. Co-Expression of a TY145 Protease Variant and CI-2A Variants and Release of Protease Activity

A Bacillus subtilis host strain was transformed with 3 copies of a protease variants having the amino acid sequence of SEQ ID NO: 5 with the substitutions S27K N109K S111E S171E S173P G174K S175P F180Y G182A L184F Q198E N199K T297P (disclosed in WO 2016/097350 and WO 2016/097354), and further transformed with 2 copies of the CI-2A variants having the amino acid sequence of the mature protein of SEQ ID NO:1 with substitutions:
(CI08) K21H+P25S+E26H+K30H+E33H; or
(CI10) K30H+E33H+K43H+P44A+P52A.

The strains were prepared essentially as described in example 2.

The fermentation broths were tested for release of protease activity at different pH values in the range of 3.0 to 6 as described in example 4. Results are shown in FIG. 4. The results show that for the fermentation broths comprising the variants of the invention the protease activity can be released at pH 4.0-4.5. At pH 3.5 and lower the protease activity completely inactivated inactivation under such harsh conditions.

1-22: (canceled)

23: A protease inhibitor having the ability to inhibit a S1 or a S8 protease at pH in the range of 6.0 to 9.0, where a complex of a S1 or S8 protease and the inhibitor dissociates when pH is lowered to a pH below 6.0.

24: The protease inhibitor of claim 23, wherein the complex of a S1 or S8 protease and the inhibitor dissociates when pH is adjusted to a pH value in the range of 4.0 to 6.0.

25: The protease inhibitor of claim 24, wherein the complex of a S1 or S8 protease and the inhibitor dissociates when pH is adjusted to a pH value in the range of 4.5 to 6.0.

26: The protease inhibitor of claim 25, wherein the complex of a S1 or S8 protease and the inhibitor dissociates when pH is adjusted to a pH value in the range of 4.5 to 5.5.

27: The protease inhibitor according to claim 23, wherein the protease inhibitor is a variant having protease inhibitor activity and having at least 60% sequence identity to the mature polypeptide of SEQ ID NO: 1, and comprising a substitution at one or more positions corresponding to positions 21, 25, 26, 30, 33, 43, 44 and 52 of SEQ ID NO: 1.

28: The variant of claim 27, wherein a complex comprising the variant and the protease having the sequence of SEQ ID NO: 2 dissociates at a higher pH compared with a complex comprising the protease inhibitor having the sequence of the mature polypeptide of SEQ ID NO: 1 and the protease having the sequence of SEQ ID NO: 2.

29: The variant of claim 28, wherein the complex comprising the variant and the protease having the sequence of SEQ ID NO: 2 dissociates at a pH value that is at least 0.4 pH units higher, where a complex comprising the protease inhibitor having the sequence of the mature polypeptide of SEQ ID NO: 1 and the protease having the sequence of SEQ ID NO: 2 dissociates.

30: The variant according to claim 27, wherein the substitutions are selected from substitutions corresponding to the following substitutions in SEQ ID NO: 1: K21H, P25S, E26H, K30H, E33H, K43H, P44A and P52A.

31: The variant according to claim 27, comprising the substitutions: K21H, P25S, E26H, K39H and E33H.

32: The variant of claim 31, further comprising one or more substitutions selected from D42N, E45G and Q47H.

33: The variant according to claim 27, comprising the substitutions:
K43H+P44A+P52A;
K21H+P25S+K30H;
K21H+P25S+E26H+K30H+E33H;
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; or
K21H+P25S+E26H+K30H+E33H+E45G+Q47H.

34: The variant of claim 33, having the sequence of SEQ ID NO: 1 with the substitutions:
K43H+P44A+P52A;
K21H+P25S+K30H;
K21H+P25S+E26H+K30H+E33H;
K30H+E33H+K43H+P44A+P52A;
K21H+P25S+E26H+K30H+E33H+D42N+E45G;
K21H+P25S+E26H+K30H+E33H+E45G;
K21H+P25S+E26H+K30H+E33H+Q47H;
K21H+P25S+E26H+K30H+E33H+D42N+Q47H; or
K21H+P25S+E26H+K30H+E33H+E45G+Q47H.

35: A polynucleotide encoding a protease inhibitor according to claim 23.

36: A plasmid, expression construct or host cell comprising the polynucleotide of claim 35.

37: A method of producing a protease inhibitor according to claim 23, the method comprising the steps of:
a. providing a host cell comprising a polynucleotide encoding a protease inhibitor according to claim 23,
b. cultivating the host cell under conditions leading to expression of the protease inhibitor, producing a fermentation broth; and, optionally
c. recovering the protease inhibitor from the fermentation broth

38: A method for producing a complex consisting of a S1 or S8 protease and a protease inhibitor according to claim 23, the method comprising the steps of:
a. providing a microorganism expressing a S1 or a S8 protease, and a microorganism expressing a protease inhibitor according to claim 23;
b. cultivating the microorganism expressing a S1 or S8 protease, and the microorganism expressing a protease inhibitor under conditions inducing the expression of the S1 or the S8 protease and of the protease inhibitor, whereby a complex consisting of the S1 or the S8 protease and the protease inhibitor is formed; and, optionally
c. recovering the complex consisting of the S1 or S8 protease and the protease inhibitor from the fermentation broth.

39: The method of claim 38, wherein the microorganism expressing the S1 or S8 protease is the same microorganism that expresses the protease inhibitor.

40: A method for producing a S1 or S8 protease, comprising the steps of:
a. providing a microorganism expressing a S1 or a S8 protease, and a microorganism expressing a protease inhibitor according to claim 38;
b. cultivating the microorganism expressing a S1 or S8 protease, and the microorganism expressing a protease inhibitor under conditions inducing the expression of the S1 or the S8 protease and of the protease inhibitor, whereby a complex consisting of the S1 or the S8 protease and the protease inhibitor is formed;
c. adjusting the pH to a low pH value where the complex of the S1 or S8 protease and the protease inhibitor dissociates and the protease activity is released, and, optionally
d. recovering the S1 or S8 protease.

41: The method of claim 40, where pH in step c. is adjusted to a pH value in the range of 4.0 to 4.5.

42: The method according to claim 40, wherein the microorganism expressing the S1 or S8 protease is the same microorganism that expresses the protease inhibitor.

43: The method according to claim 40, wherein step c. is performed before the recovery process, during the recovery process or after the complex consisting of the S1 or the S8 protease and the protease inhibitor has been recovered.

44: The method according to claim 16, wherein the S1 or the S8 protease is selected from polypeptides having protease activity and having at least 60% sequence identity to any one of SEQ ID NO: 2; SEQ ID NO: 3; SEQ ID NO: 4, SEQ ID NO: 5 and SEQ ID NO: 6.

45: The method according to claim 18, wherein the S1 or the S8 protease is selected from polypeptides having protease activity and having at least 60% sequence identity to any one of SEQ ID NO: 2; SEQ ID NO: 3; SEQ ID NO: 4, SEQ ID NO: 5 and SEQ ID NO: 6.


SEQUENCE LISTING (6 sequences)

SEQ ID NO: 1 - protein, 83 aa, Hordum vulgaris
SSVEKKPEGVNTGAGDRHNLKTEWPELVGKSVEEAKKVILQDKPEAQIIVLPVGTIVTMEYRIDRVRLFVDKLDNIAQVPRVG

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 275 aa, Bacillus amyloliquefaciens [sequence omitted]

SEQ ID NO: 4 - protein, 274 aa, Bacillus sp.
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 5 - protein, 311 aa, Bacillus sp. [sequence omitted]

SEQ ID NO: 6 - protein, 188 aa, Nocardiopsis sp.
ADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT